(19) United States
(12) Patent Application Publication
(10) Pub. No.: US 2021/0068999 A1
(43) Pub. Date: Mar. 11, 2021

(54) APPETITE CONTROL DEVICE

(71) Applicant: Kendall Ann Whitney, Westlake Village, CA (US)

(72) Inventor: Kendall Ann Whitney, Westlake Village, CA (US)

(21) Appl. No.: 16/951,439

(22) Filed: Nov. 18, 2020

Related U.S. Application Data
(63) Continuation-in-part of application No. 16/341,670, filed on Apr. 12, 2019.
(60) Provisional application No. 62/938,574, filed on Nov. 21, 2019.
(60) Provisional application No. 62/407,128, filed on Oct. 12, 2016.

(51) Int. Cl. A61F 5/00; A23L 33/10; A24B 15/24; A24F 47/00; G16H 20/60; G16H 40/67; G16H 50/20

(52) U.S. Cl. CPC A61F 5/0006; A23L 33/10; A24B 15/243; A23V 2002/00; G16H 20/60; G16H 40/67; G16H 50/20; A24F 47/00

(57) ABSTRACT

Various embodiments of the present invention provide a method and a system. The system having an appetite control device for covering a plurality of teeth, wherein the appetite control device comprises an arcuate shape having an outer sidewall, an inner sidewall and a top wall connecting the inner sidewall and outer sidewall to form a channel, wherein the channel is configured for receiving a plurality of teeth, wherein the appetite control device includes a larger width at ends and a narrower width at a front. The system further includes a user device having a memory and a processor configured to execute executable instructions stored in the memory to receive data associated with usage of the appetite control device and determine an output data associated with appetite information of the user based on analysis of the received data using a pre-trained machine learning model.

### CROSS-REFERENCE TO RELATED APPLICATION

This application is a continuation-in-part of U.S. Non-Provisional patent application Ser. No. 16/341,670, filed Apr. 12, 2019, which is a 35 U.S.C. § 371 National Stage Entry of International Application No. PCT/US2017/056381, filed Oct. 12, 2017, which claims the priority benefit of U.S. Provisional Patent Application Ser. No. 62/407,128, filed Oct. 12, 2016, and this application claims the priority benefit of U.S. Provisional Patent Application Ser. No. 62/938,574 filed Nov. 21, 2019, all of which are incorporated herein by reference in their entirety for all purposes.

### TECHNOLOGICAL FIELD

Example embodiments of the present invention generally relate to health aides and more particularly relate to a dieting device.

### BACKGROUND

Appetite is the desire in a person to eat food. Appealing foods can stimulate appetite even when hunger is absent thereby causing disturbance to normal eating routine. The disturbance in normal eating schedule results in change of metabolism in the person. Due to the change in metabolism, the person may experience various eating disorders such as over-eating, irregular eating, excessive or subdued hunger, or the like. The persons having obesity are prone to a plurality of diseases such as heart disease, stroke, diabetes, sleep apnea, digestive problems, gynecological problems or the like.

Existing ways to lose weight includes dieting, consuming diet pills, performing exercise or undergoing a weight loss surgery. The existing methods provide limited gains, for example, in the case of dieting, people attempt dieting but due to lack of efficient tracking, appetite information is unavailable. Therefore, many people are not able to pursue dieting continuously resulting in minimal success. Some people attempt weight loss surgery such as gastric bypass, but they are not guaranteed to result in weight loss. Further, none of the existing ways tackle the root cause of the problem that is controlling the appetite. Controlling the appetite would essentially mean changing the eating habit and ensuring behavioral change of the person.

### SUMMARY

According to some embodiments, a system for providing appetite information of a user is disclosed. The system includes an appetite control device for covering a plurality of teeth. The appetite control device comprises an arcuate shape having an outer sidewall, an inner sidewall and a top wall connecting the inner sidewall and outer sidewall to form a channel. The channel is configured for receiving a plurality of teeth, where the appetite control device comprises a larger width at ends and a narrower width at a front of the appetite control device. The system includes a user device comprising a memory configured to store executable instructions and a processor configured to execute the executable instructions stored in the memory. The processor is configured to receive data associated with usage of the appetite control device. The processor is configured to determine, an output data associated with appetite information of the user based on analysis of the received data using a pre-trained machine learning model. The processor is further configured to provide the output data on a Graphical User Interface (GUI) of the user device.

According to some embodiments, the appetite control device may include one or more apertures on at least one of the outer side wall, the inner side wall, and the top wall.

According to some embodiments, the appetite control device may include a flavour pouch comprising one or more flavour tabs.

According to some embodiments, the flavour pouch includes a top surface and a bottom surface to hold the one or more flavour tabs.

According to some embodiments, the flavour pouch includes a plurality of apertures with varying sizes.

According to some embodiments, the flavour pouch is made from a mesh to hold the one or more flavour tabs.

According to some embodiments, the flavour pouch is attached to the appetite control device by one or more attachment fasteners.

According to some embodiments, the flavour pouch includes surface indicia to provide feedback to the user.

According to some embodiments, the appetite control device includes a front portion of the outer side wall to cover a part of front portion of the plurality of teeth of the user.

According to some embodiments, the appetite control device includes a portion of the top wall to cover a part of top portion of the plurality of teeth of the user.

According to some embodiments, the portion includes one or more of a hook or a J shaped portion to secure against the plurality of teeth of the user.

According to some embodiments, the appetite control device includes an upper appetite control device to cover upper teeth of the user and a lower appetite control device to cover lower teeth of the user, where the lower appetite control device is identical to the upper appetite control device.

According to some embodiments, the appetite control device is made of mouldable material to ergonomically align with the plurality of teeth of the user.

According to some embodiments, the appetite control device includes a plurality of sensors to provide the data associated with usage of the appetite control device to the user device.

According to some embodiments, the processor is further configured to provide gamification elements, via the GUI, to the user based on the received data.

According to some embodiments, the system further includes a server, where the server is one or more of a physical server or a cloud server.

According to some embodiments, the machine learning model is processed in one or more of: the user device, the physical server, or the cloud server.

According to some embodiments, a computer-implemented method for providing appetite information of user is disclosed. The computer-implemented method includes receiving by a user device data associated with usage of an appetite control device. The computer-implemented method includes determining an output data associated with appetite information of the user based on analysis of the received data using a pre-trained machine learning model. The computer-implemented method further includes providing the output data on a Graphical User Interface (GUI) of the user device.

According to some embodiments, the computer-implemented method includes receiving data associated with usage of the appetite control device further comprises receiving the data from a plurality of sensors associated with the appetite control device to provide the data associated with usage of the appetite control device to the user device.

According to some embodiments, the computer-implemented method further includes providing gamification elements, via the GUI, to the user based on the received data.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows a top view of an exemplary appetite control device, in accordance with an example embodiment of the present invention;

FIG. 2 shows an exemplary appetite control device having a flavor pouch, in accordance with an example embodiment of the present invention;

FIG. 3 shows a perspective view of the appetite control device in FIG. 1, in accordance with an example embodiment of the present invention;

FIG. 4 shows a cross-sectional view of the appetite control device in FIG. 1 about line G-G, in accordance with an example embodiment of the present invention;

FIG. 5 shows a bottom view of the appetite control device in FIG. 1, in accordance with an example embodiment of the present invention;

FIG. 6 shows a top view of an appetite control device having a flavor pouch, in accordance with an example embodiment of the present invention;

FIG. 7 shows a perspective view of an appetite control device having a flavor pouch in accordance with an example embodiment of the present invention;

FIG. 8A shows a shows a perspective view of an appetite control device having a flavor pouch and covering a minimal number of teeth, in accordance with an example embodiment of the present invention;

FIG. 8B shows a perspective view of an appetite control device covering a minimal number of teeth, in accordance with an example embodiment of the present invention;

FIG. 8C shows a perspective view of an appetite control device covering a lower portion of a user's front teeth, in accordance with an example embodiment of the present invention;

FIG. 8D shows a perspective view of an appetite control device covering a lower portion of a user's back teeth, in accordance with an example embodiment of the present invention;

FIG. 8E shows a view of an appetite control device covering a lower portion of a user's front teeth, in accordance with an example embodiment of the present invention;

FIG. 8F shows a view of an appetite control device covering a lower portion of a user's front teeth, in accordance with an example embodiment of the present invention;

FIG. 8G shows a perspective view of an appetite control device covering a user's back teeth in accordance with an example embodiment of the present invention;

FIG. 9 shows a perspective view of an appetite control device having a flavor pouch with a plurality of apertures in accordance with an example embodiment of the present invention,

FIG. 10 shows perspective view of an appetite control device having a flavor pouch made from a mesh, in accordance with an example embodiment of the present invention;

FIG. 11 shows perspective view perspective view of an appetite control device having a detachable flavor pouch with the detachable flavor pouch detached, in accordance with an example embodiment of the present invention;

FIG. 12 shows a perspective view of the appetite control device of FIG. 11 having the detachable flavor pouch attached, in accordance with an example embodiment of the present invention;

FIG. 13 shows a perspective view of an appetite control device having a flavor pouch with surface indicia, in accordance with an example embodiment of the present invention;

FIG. 14 shows a perspective view of an example appetite control device having a plurality of apertures, in accordance with an example embodiment of the present invention;

FIG. 15 shows an appetite control device system with one or more connected devices, in accordance with an example embodiment of the present invention;

FIG. 16 shows a high-level block diagram of an appetite control device system, in accordance with an example embodiment of the present invention;

FIG. 17 shows a high-level block diagram and process of a computing system, in accordance with an example embodiment of the present invention;

FIG. 18 shows a block diagram and process of an exemplary system in which an embodiment may be implemented;

FIG. 19 shows a cloud computing environment for implementing an embodiment of the system and method disclosed, in accordance with an example embodiment of the present invention; and

FIG. 20 shows a computer implemented method for providing appetite information of user, in accordance with an example embodiment of the present invention.

### DETAILED DESCRIPTION

In the following description, for purposes of explanation, numerous specific details are set forth in order to provide a thorough understanding of the present invention. It will be apparent, however, to one skilled in the art that the present invention may be practiced without these specific details.

Throughout the following description, numerous references may be made regarding servers, services, or other systems formed from computing devices. It should be appreciated that the use of such terms is deemed to represent one or more computing devices having at least one processor configured to or programmed to execute software instructions stored on a computer readable tangible, non-transitory medium or also referred to as a processor readable medium. For example, a server can include one or more computers operating as a web server, data source server, a cloud computing server, a remote computing server or other type of computer server in a manner to fulfill described roles, responsibilities, or functions. Within the context of this document, the disclosed modules are also deemed to comprise computing devices having a processor and a non-transitory memory storing instructions executable by the processor that cause the device to control, manage, or otherwise manipulate the features of the devices or systems.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present invention. Further, it is to be understood that the phraseology and terminology employed herein are for the purpose of the description and should not be regarded as limiting. Any heading utilized within this description is for convenience only and has no legal or limiting effect.

Various embodiments provide system and method for providing appetite control information of a user. The system and method comprises an appetite control device that may be worn by placing the device in the mouth on all, or part of the upper and/or lower teeth, by a user. The appetite control device is a physical impediment to eating and/or snacking between meals as it must be removed prior to eating. The appetite control device also makes the user think about not eating by making the user more aware of their behavior. In order to eat food, or snack between meals, the user must consciously remove the appetite control device from their teeth prior to snacking. The user then consciously places the upper and/or lower appetite control devices back in their mouth after eating and/or snacking. In some embodiments, the appetite control device may contain a flavor pouch. The flavor pouch may receive a flavored tab such as mint, lemon, cherry, or bubblegum. The appetite control device may be stored in a compact case when not in use. The appetite control device may be used in conjunction with diet and exercise, without negative consequences or side effects. While other weight loss techniques may focus on fewer calories in foods, diet pills, and/or weight loss surgery, these techniques do not address the behavioral component in weight loss, which is addressed by the use and design of the embodiments of an oral appliance comprising an appetite control device disclosed herein. The appetite control device is a behavioral modification tool that makes users more conscious of what they eat. Users may decide when to use the appetite control device, such as for appetite control, smoking cessation, etc. In one embodiment, the appetite control device is small and discrete, and does not alert others when the user is wearing the appetite control device and/or dieting. Users can continue to speak and drink without impediment while wearing the appetite control device. Further, the appetite control device tracks data on usage and provides the data to a user device of the system. The system may determine an output using a pre-trained machine learning model. Further, the system may provide appetite information of the user based in the determined output. The appetite information ensures efficient tracking of the user's appetite and provides recommendation to the user for maintaining a healthy appetite.

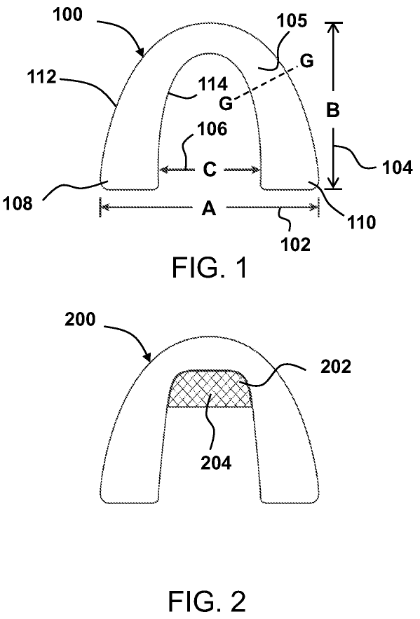

FIG. 1 illustrates a top view of a system comprising an appetite control device 100 for covering teeth, in accordance with an example embodiment of the present invention. The appetite control device may be an oral appliance. The appetite control device 100 disclosed herein comprises an overall arcuate shape (e.g., U-shaped) having an outer sidewall 112, an inner sidewall 114, and a top wall 105 connecting the inner sidewall 114 and outer sidewall 112 to form a channel (not shown in FIG. 1). The channel is configured for receiving at least a portion of a plurality of teeth such that the appetite control device 100 comprises a larger width at ends and a narrower width at a front of the appetite control device. The appetite control device 100 may have an outer width A 102, a height B 104, and an inner width C 106 between left end 108 and right end 110. In some embodiments, the appetite control device 100 may have the outer width A 102 between 5 cm-6 cm, the height B 104 between 5 cm-6 cm, and the inner width C 106 between 3 cm-4 cm. In a preferred embodiment, the appetite control device may have the outer width A 102 of about 5.715 cm, the height B 104 of about 5.08 cm, and the inner width C 106 of about 3.175 cm. In some embodiments, the channel of the appetite control device 100 may be wider ends 108 and 110 by the location of the back molars of the user. In other embodiments, the appetite control device 100 may contain one or more apertures 1402 (shown in FIG. 14) on the outer sidewall 112, one or more apertures 1404 (shown in FIG. 14) on the inner sidewall 114, and/or one or more apertures 1406 (shown in FIG. 14) on the top wall 105 for ventilation, saliva flow, cleaning, and/or drying. The appetite control device 100 may comprise an upper appetite control device to cover upper teeth of the user and a lower appetite control device to cover lower teeth of the user. The upper appetite control device and the lower appetite control device are identical. Further, the upper and/or lower appetite control device may contain one or more apertures on an outer sidewall 112 and/or an inner sidewall 114.

In one embodiment, the appetite control device 100 comprises moldable material that is ergonomically formed for alignment with the bite of the user. The appetite control device 100 may comprise essentially rigid material such as heat cured acrylic resin, a hard, clear plastic material. The appetite control device 100 may also comprise essentially soft material such as heat-contoured laminate material. In one embodiment, the appetite control device 100 comprises dual laminate material, such as hard acrylic on the outside and soft laminate material on the inside. In another embodiment, the appetite control device 100 comprises a thermo plastic material adapted to fit a user's teeth and gums by heating and molding such as boiling then placing in the mouth. The appetite control device is placed in hot water to soften, then placed in the mouth and shaped around the teeth using finger and tongue pressure. Retention mechanisms such as fins may be included in designated areas which allow increasing retention force on the teeth. In an additional embodiment the appetite control device 100 comprises Ethylene-vinyl acetate. An impression of the user's teeth may be used to create a properly fitting appetite control device 100. The impression may be obtained using an impression and dental putty. The appetite control device 100 reduces sudden eating instincts of the user by utilizing a flavor pouch (not shown in FIG. 1) as shown in FIG. 2.

FIG. 2 shows an exemplary appetite control device 200 having a flavor pouch 202 for covering all or a portion of the upper teeth. The appetite control device 200 can similarly be configured for use on lower teeth. The appetite control device 200 may be identical to the appetite control device 100 of FIG. 1, except for the addition of the flavor pouch 202. The flavor pouch 202 may be used to hold one or more flavor tabs 204 and/or flavor gels. The flavor tabs 204 may be inserted into the flavor pouch 202 and provide one or more flavors, such as mint, cherry, or bubblegum, to a user through the roof of their mouth.

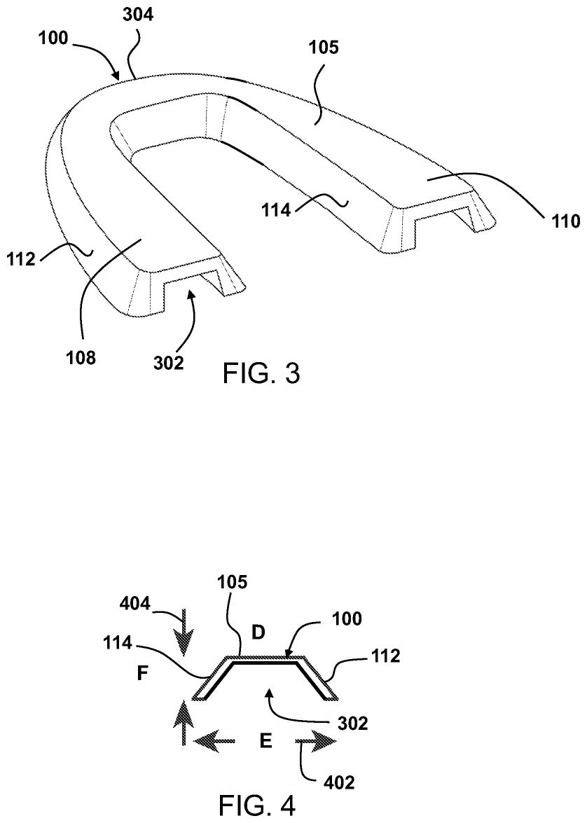

FIG. 3 shows a perspective view of the appetite control device 100 in FIG. 1. A channel 302 of the appetite control device 100 may be formed by the outer sidewall 112, inner sidewall 114, and top wall 105. The appetite control device includes a front 304 portion and ends 108, 110.

FIG. 4 shows a cross-sectional view of the appetite control device 100 in FIG. 1 about line G-G. The appetite control device 100 may have a generally trapezoidal cross section such as an isosceles trapezoid, forming said channel 302. In one example, the appetite control device 100 may have a lower width D, an upper width E 402, and a height F 404. In some embodiments, the appetite control device 100 may have the lower width D between 0.1 cm-1 cm, the upper width E 402 between 1 cm-2 cm, and the height F 404 between 0.1 cm-1 cm. In a preferred embodiment, the appetite control device may have the lower width D of about 0.635 cm, the upper width E 404 of about 1.27 cm, and the height F 404 of about 0.47625 cm. In some embodiments, the appetite control device 100 may be thinner. In some embodiments, other example dimensions different than trapezoidal dimension are possible. The dimensions of the appetite control device can be configured based on the oral characteristics such as mouth size, teeth size and position, or the like to comfortably fit in the mouth of a user.

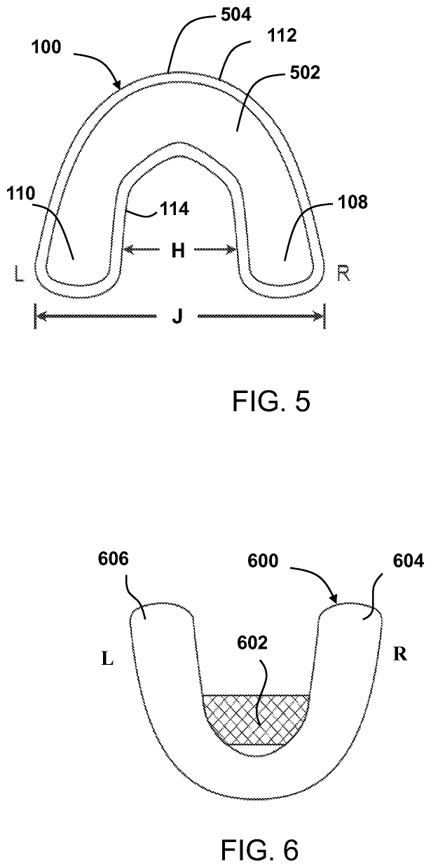

FIG. 5 shows a bottom view of the appetite control device 100 having a left side 110 and a right side 108. The appetite control device 100 may have the outer sidewall 112 and the inner sidewall 114. In some embodiments, the sidewalls 112, 114 may surround a perimeter of the appetite control device 100. The sidewalls 112, 114 may have a suitable range of dimensions, to cause the appetite control device 100 to ergonomically align with the user's teeth. In some embodiments, the appetite control device 100 may have an inner width H between 3 cm-4 cm, and an outer width J between 5 cm-6 cm. In a preferred embodiment, the inner width H may be 3.175 cm and the outer width J may be 5.715 cm. The upper and/or lower appetite control device may have an overall U-shape with the outer sidewall 112 and inner sidewall 114 surrounding a channel 502 for receiving teeth of a user. The upper and/or lower appetite control device may have a larger width at ends 108, 110 and a narrow width at front 504. The thickness of the sidewalls 112, 114 and the top wall 105 forming the channel 502 may be minimal (e.g., between 0.25 mm to 3 mm, and preferably between 0.5 mm to 1 mm) compared to a conventional mouth guard, sufficient to maintain the appetite control device in the user's mouth while engaging a portion of the teeth, and inhibit eating or drinking rather than to protect the teeth from grinding, impact, etc. Other example dimensions are possible. The dimensions of the appetite control device can be configured based on the oral characteristics, mouth size, teeth size and position, or the like to comfortably position the appetite control device 100 in the mouth of a user.

FIG. 6 shows another embodiment of an appetite control device 600 having a flavor pouch 602. The appetite control device 600 may have a left side 606 and a right side 604 denoted by L and R respectively. In some embodiments, the appetite control device may be used as an upper and/or lower appetite control device, and may be used as an anti-smoking device and/or a deterrent to smoking. The flavor pouch 602 may be used to hold one or more nicotine tabs and/or nicotine gels that deliver nicotine to the user. The appetite control device 600 provides nicotine flavor to the user whenever the user feels like smoking. Therefore, the appetite control device 600 prevents the user from smoking by providing the nicotine flavor to the user's mouth.

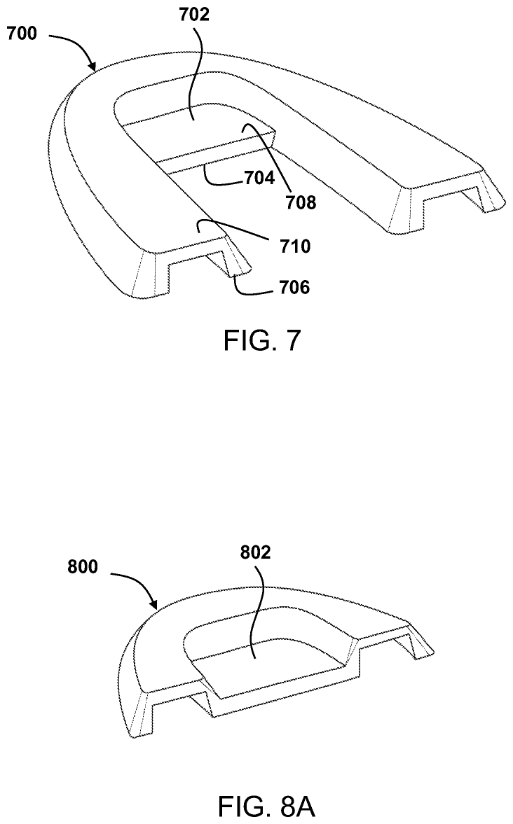

FIG. 7 shows a perspective view of an example upper (or lower) appetite control device 700 having a flavor pouch 702. The appetite control device 700 may be identical to the appetite control device 100, with the addition of the flavor pouch 702. The flavor pouch 702 is shown as having a top surface 704 substantially level with an upper surface 706 of the upper appetite control device 700 so as to be in contact with roof of a wearer's i.e., user's mouth. In some embodiments, a lower surface 708 of the flavor pouch 702 may be substantially level with a lower surface 710 of the upper appetite control device 700. In other embodiments, the flavor pouch 702 may be disposed between the upper surface 706 and lower surface 710 of the upper appetite control device 700.

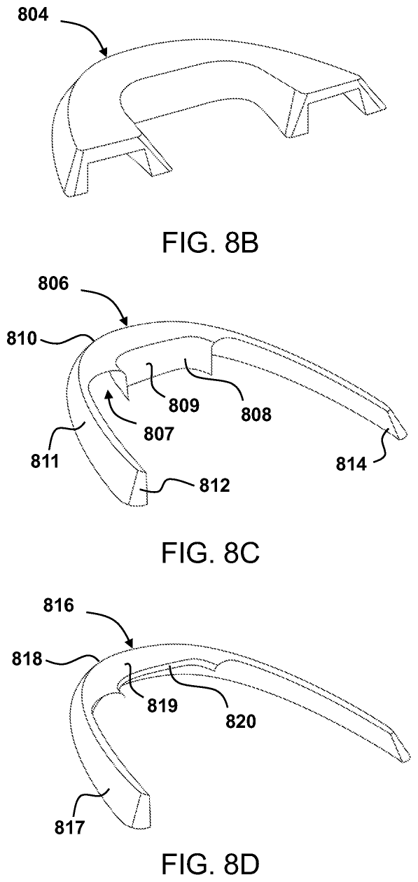

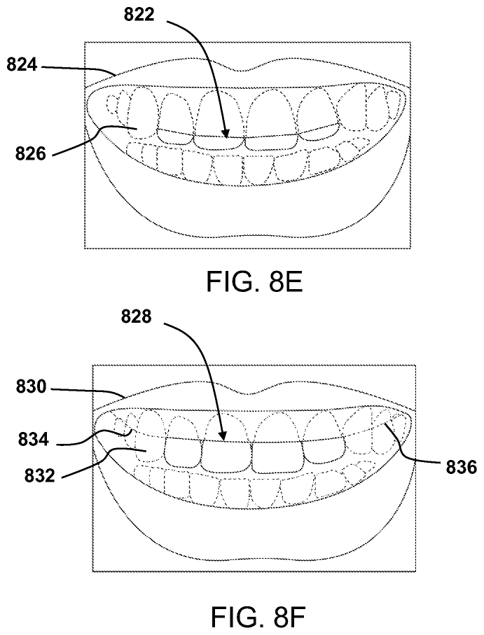

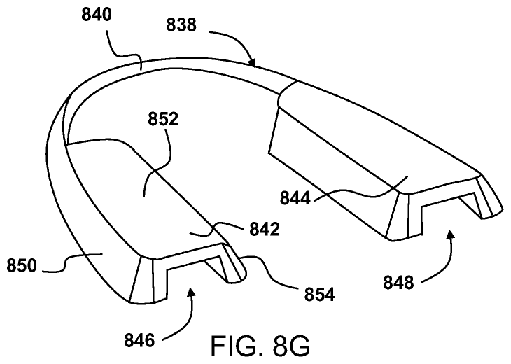

The appetite control device 100 may be configured to cover various portions of a user's teeth. The various configurations of the appetite control device 100 are shown in FIG. 8. The various configurations enable the appetite control device 100 to be flexible and with reduced cross-sectional area.

FIG. 8A shows a perspective view of an upper (or lower) appetite control device 800 having a flavor pouch 802 and covering a minimal number of teeth. The appetite control device 800 may only extend to cover the front upper teeth of a wearer needed to support the flavor pouch 802 disposed between the ends of the upper appetite control device 800. In some embodiments, the upper appetite control device 800 may be constructed from a transparent material, e.g., a clear plastic, so as to minimize the visual appearance of the upper appetite control device 800. The upper appetite control device 800 may be minimal in construction so as to provide protection to a user's teeth.

FIG. 8B shows a perspective view of an upper (or lower) appetite control device 804, without a flavor pouch, covering a minimal number of teeth. The upper appetite control device 804 may be used to cover only three or four teeth and is configured to act as an impediment to eating or drinking to prevent grinding or protect teeth from impact. When placed in the mouth of a user, when compared to the device 700 in FIG. 7, the device 800 in FIG. 8A does not extend to the molar teeth, and covers the front and canine teeth.

FIG. 8C shows a perspective view of an appetite control device 806 covering a lower portion of a user's front teeth, in accordance with an example embodiment of the present invention. A front portion 810 of the appetite control device 806 may only cover a portion of the user's front teeth, as depicted in FIG. 8E. The channel 807 defined by the walls 809, 811 of the appetite control device 806 cover the front teeth of the user, such that the device 806 is supported at the mouth over the user's front teeth via a back portion 808 of the inner sidewall 809. The outer sidewall 811 of the appetite control device 806 may extend to the back of a user's mouth along the gum line next to molars via ends 812, 814, to provide more stability in the user's mouth. Ends 812, 814 may be constructed from plastic, metal, a wire, etc. and may prevent the appetite control device 806 from being swallowed, keep the device 806 in place in the mouth, or the like. In some embodiments, ends 812, 814 may not be present.

FIG. 8D shows a perspective view of an appetite control device 816 covering a lower portion of a user's back teeth via back portion 820. The appetite control device 816 may include a front portion 818 of an outer sidewall 817 that covers at least a part of the front of a user's teeth and the back portion 820 of the top wall 819 that covers only a portion of the top of a user's teeth, such that the tongue may freely touch the back of the front teeth inside the mouth for easier speech making. In one embodiment, the portion 820 may have a hook, or J, shape to secure against the teeth of a user without inhibiting the user's ability to use their tongue and talk normally. Accordingly, a user may be able to wear the appetite control device 816 without notifying other people that they are using it as their speech will not be inhibited thereby ensuring privacy.

FIG. 8E shows a view of an upper appetite control device 822 covering a lower portion of a user's front teeth 826. The upper appetite control device 822 may be inserted into a mouth 824 of the user and only cover a lower portion of the users front teeth 826, such that the visual appearance of the upper appetite control device 822 is minimized.

FIG. 8F shows a view of an appetite control device 100 covering a lower portion of a user's front teeth, in accordance with an example embodiment of the present invention. The appetite control device 100 is an upper appetite control device 828. The upper appetite control device 828 may be inserted into a mouth 830 of a user to cover at least a portion of front of the user's teeth 832. The back of the user's teeth may be minimally covered in a hook, or J shape so as to not inhibit the user's tongue so that the user may pronounce words normally. The upper appetite control device 828 may include one or more ends 834, 836 shown in dashed lines that may extend to the back of a user's mouth and/or cover additional teeth 832. The ends 834, 836 may be plastic, metal, a wire, etc. and may be used to hold the upper appetite control device 828 in place and/or prevent swallowing the upper appetite control device 828. The upper appetite control device 828 may be used in conjunction with a lower appetite control device, such as shown in FIG. 1. In some embodiments, only a lower appetite control device may be used to minimize visual appearance to others, while still inhibiting drinking and/or eating. The upper appetite control device and lower appetite control device may be identical in some embodiments. The upper appetite control device and lower appetite control device may be different in some embodiments.

FIG. 8G shows a view of an upper appetite control device 838 covering a user's back teeth. The appetite control device 838 may include an outer wall 850, an inner wall 854, and a top wall 852 forming two channel segments 846, 848, connected by a portion 840 of the outer wall 850. The outer wall 850 may be spaced between an outside surface of a user's molars and a cheek of a user. The inner wall 854 may be spaced between an inner surface of a user's molars and an inside of a mouth of a user. The top wall 852 may be spaced between the top surfaces of the user's molars. The channel segments 846, 848 may surround portions of the user's molars. The front portion 840 of the outside channel 850 may extend to connect the ends 842, 844 of the appetite control device 838. The front portion 840 may be a thin segment which may be disposed between the gums and inner lip of a user where it may not be readily visible to an outside observer. In other embodiments, the front portion 840 may extend to cover a portion of the user's front teeth. The appetite control device may be formed to cover a portion of any of a user's teeth with varying degrees of visibility to an outside observer based on a desired ease of wearing, ease of speaking, ability to prevent eating and/or drinking, or the like.

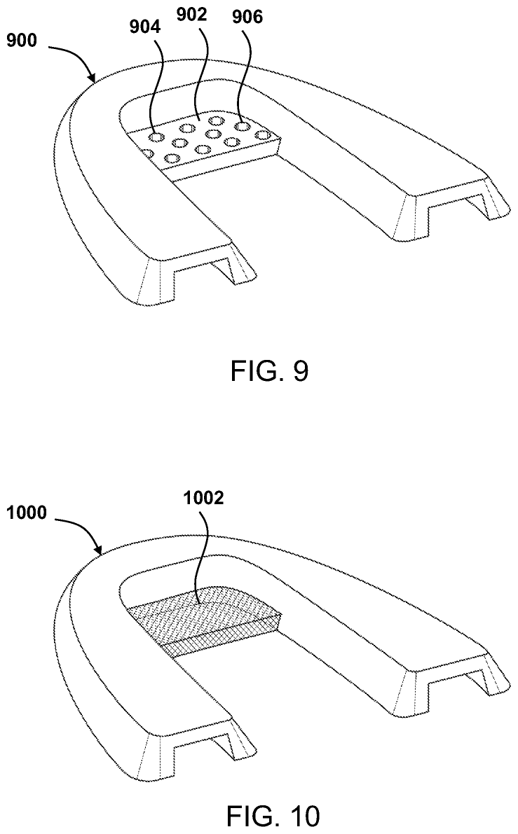

FIG. 9 shows a perspective view of an appetite control device 900 having a flavor pouch 902 with a plurality of apertures, 904, 906 in accordance with an example embodiment of the present invention. The flavor pouch 902 may have the apertures 904, 906 on a top surface, a bottom surface, and/or a side. The apertures 904, 906 may be an opening or a hole that provides the flavor from the flavor pouch 902 to the user's mouth. In some embodiments, the aperture sizes may vary based on the desired longevity of a tab and/or gel placed in the flavor pouch 902.

FIG. 10 shows perspective view of an appetite control device 1000 having a flavor pouch made from a mesh, in accordance with an example embodiment of the present invention. The appetite control device 1000 includes the flavor pouch made from a mesh 1002. The mesh 1002 may be used to hold a tab and/or flavored gel in a desired position in the user's mouth relative to the upper appetite control device 1000 while allowing the tab to dissolve through the mesh and provide flavor to the user.

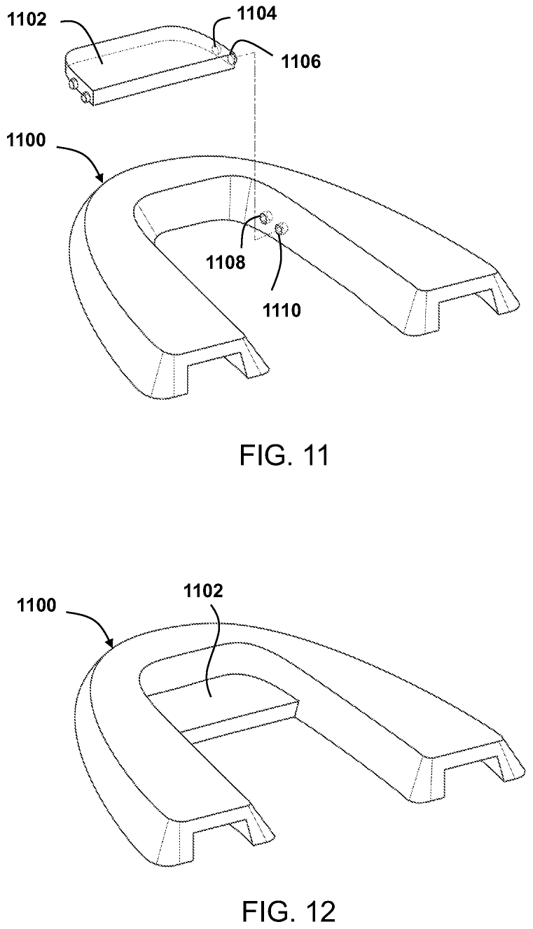

FIG. 11 shows a perspective view of an upper (or lower) appetite control device 1100 having a detachable flavor pouch 1102 with the detachable flavor pouch 1102 detached. The upper appetite control device 1100 may have one or more detachable attachment fasteners 1108, 1110, such as indentations, hooks, etc. The detachable flavor pouch 1102 may have one or more corresponding fasteners 1104, 1106, such as protrusions, hooks, indentations, etc. for receiving the one or more fasteners 1108, 1110 on the upper appetite control device 1100. The detachable flavor pouch 1102 may be reusable and a tab and/or gel may be inserted into the flavor pouch 1102. In other embodiments, the detachable flavor pouch 1102 may be disposable and/or dissolve during use. A user may utilize a disposable detachable flavor pouch 1102 and then replace with another disposable detachable flavor pouch 1102 once the first pouch has dissolved. The detachable flavor pouch 1102 may include any of the designs, elements, and/or functions as shown in FIGS. 7-10 and 13.

FIG. 12 shows a perspective view of the upper (or lower) appetite control device 1100 of FIG. 11 having the detachable flavor pouch 1102 attached. The one or more detachable attachment fasteners 1108, 1110 (not shown in Figure) connects the flavor pouch 1102 with the appetite control device 1100.

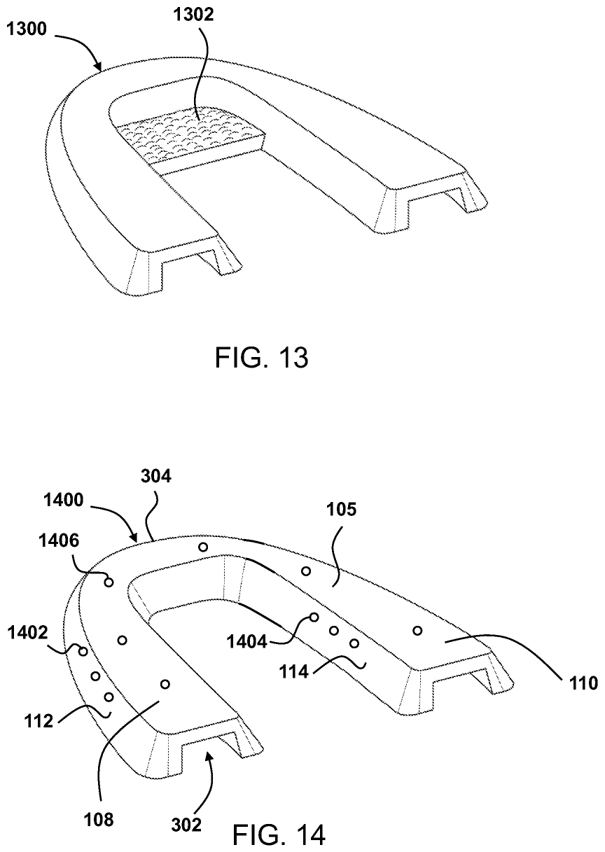

FIG. 13 shows perspective view of an appetite control device having a flavor pouch with surface indicia 1302, in accordance with an example embodiment of the present invention. The appetite control device is an upper appetite control device 1300 having a flavor pouch with surface indicia 1302. The flavor pouch may have indicia 1302 such as a surface texture. The surface indicia 1302 may be present on a top surface, a bottom surface, and/or side surfaces of the flavor pouch. The surface indicia 1302 may provide feedback to a user, via a tongue and/or roof of a mouth, when a tab enclosed within the flavor pouch, and/or a gel enclosed within the flavor pouch needs to be replaced. The surface indicia 1302 may also make the user more aware of the presence of the upper appetite control device 1300 and act to decrease their likelihood of overeating, smoking, or the like.

FIG. 14 shows a perspective view of an example appetite control device 1400 having one or more apertures, in accordance with an example embodiment of the present invention. The appetite control device 1400 may contain one or more apertures 1402 on the outer sidewall 112, one or more apertures 1404 on the inner sidewall 114, and/or one or more apertures 1406 on the top wall 105 for ventilation, saliva flow, cleaning, and/or drying. An upper and/or lower appetite control device 1400 may also contain one or more apertures 1402 on the left end 108, the right end 110 and/or the front portion 304. The appetite control device 1400 further includes the channel 302 formed by the outer sidewall 112, the inner sidewall 114, and the top wall 105.

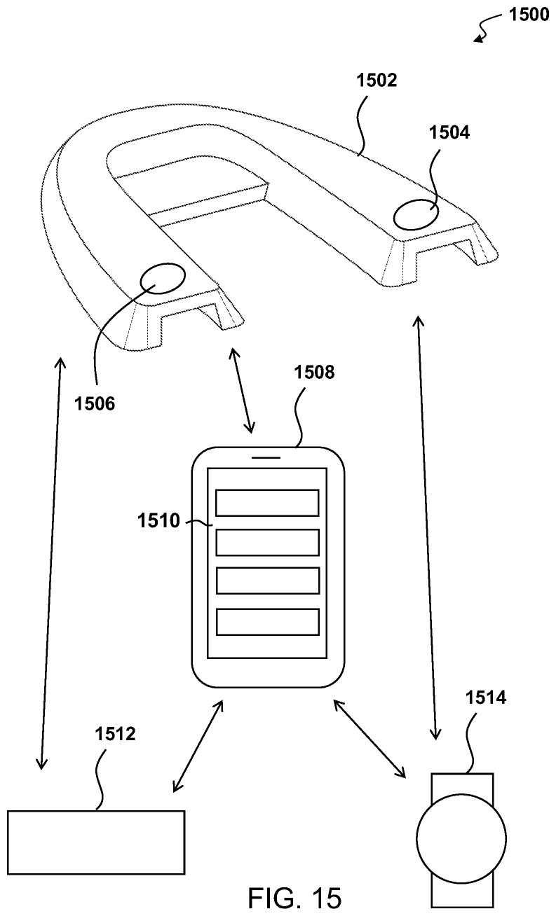

FIG. 15 shows a system 1500 with one or more connected devices, in accordance with an example embodiment of the present invention. The system 1500 includes an appetite control device 1502, electronics 1504, 1506, a user device 1508, a Graphical User Interface (GUI) 1510, a cloud server 1512, and a smart device 1514. The appetite control device 1502 may include electronics i.e., plurality of sensors 1504, 1506 for sensing data, recording data, processing data, sending data, and/or receiving data. The plurality of sensors (1504, 1506) may record data associated with usage of the appetite control device 1502 such as information on time worn, time in movement, amount of movement, any ‘chewing’ or ‘grinding’ movements, number of uses, liquid detection, food detection, and the like. In some embodiments, only one data type may be recorded. In other embodiments, two or more data types may be recorded. The system 1500 may receive, data associated with usage of the appetite control device. The received data may contain information about when the appetite control device 1502 was worn by a user and when the appetite control device 1502 was not used by the user. The duration of time the appetite control device 1502 was worn by the user may be obtained from movement data captured by the plurality of sensors (104, 1506). As the user wearing the appetite control device 1502 may rarely be perfectly still, the duration of time worn by the user can be accurately obtained. The data associated with usage of the appetite control device 1502 may be provided by the plurality of sensors 1504, 1506 to the user device 1508 of the system 1500. The system 1500 may perform analysis on the received data by using a pre-trained Machine Learning (ML) model. In an embodiment, the system 1500 may anonymize user data and transmit the anonymized user data to a cloud server 1512. The cloud server 1512 may obtain the anonymized user data from multiple users and perform analysis using the pre-trained ML model. The cloud server 1512 may provide a recommendation to the user, based on the analysis. The recommendation to the user may be one or more of alerts or prompts or timing information that ensures the user wears the appetite control device 1502 The system 1500 may then determine an output data associated with appetite information of the user based on analysis of the received data using the pre-trained ML model. The system 1500 may provide the output data on a Graphical User Interface (GUI) 1510 of the user device 1508. The user device 1508 may provide the output data to the user in one or more of a notification such as a push notification, an alarm, a vibration or the like to alert the user to wear the appetite control device 1502.

In some embodiments, the ML model performs the analysis using user's historical appetite information using deterministic algorithm, stochastic algorithm or combination thereof, such as, Bayesian network, neural network, fuzzy logic, and the like. The analyzed historical appetite information may be used by the ML model to further learn pattern from the analyzed historical appetite information. The pattern may be learnt based on one or more ML models that may include, but not limited to, a Convolutional Neural Network (CNN) model, a Hidden Martrkov Model (HMM), or a Recurrent Neural Network (RNN) model. In particular, the one or more ML models may learn the pattern based on pattern recognition algorithms. The pattern recognition algorithms may include classification methods, clustering methods, ensemble learning methods, multi-linear space learning methods, regression methods, labeling methods or the like. The pattern recognition algorithms may help in identifying behavior and trends of user's appetite information including but not limited to information about user's eating habits, eating and snacking times, one or more health conditions, preferred food items, prescribed food items and the like. The identified behavior and trends of the appetite information may be used for providing output data in the form of display of information on the GUI 1510, physical appetite control device notifications like vibration alert, light alert and the like. The output data may be provided by using the plurality of sensors 1504, 1506.

In some embodiments, the plurality of sensors 1504, 1506 may provide feedback to a user, such as via a vibration or light to inform the user that the appetite control device 1502 may be removed or should be put back in.

In some embodiments, the appetite control device 1502 is configured to record additional information relating to user habits and use of the appetite control device 1502 by the user. The appetite control device 1502 may communicate with the user device 1508, such as a smartphone; an additional connected smart device 1514, such as a smart watch or smart scale; and/or a cloud server 1512. The appetite control device 1502 may communicate via Bluetooth, Wi-Fi, RFID, a physical connection such as USB, or the like. In some embodiments, the appetite control device 1502 may communicate with the user device 1502 and the user device 1502 may receive and/or send additional data from and/or to the connected smart device 1514 and/or the cloud server 1512.

In some embodiments, the appetite control device 1502 may transmit and receive data. In other embodiments, the appetite control device 1502 may only transmit data. In additional embodiments, the appetite control device 1502 may not include any electronics and actions for the appetite control device 1502 may be confirmed by a user on the user device 1508. The user device 1508 may be a smartphone or other smart device having the GUI 1510. The GUI 1510 may also provide input options to a user of the appetite control device 1502, provide output data associated with the appetite information of the user, provide output data related to feedback on use of the appetite control device 1502, connect with additional smart devices 1514, connect with a cloud server 1512, or the like.

In some embodiments, the system 1500, via the GUI 1510 may provide the output data in the form of an alarm function, such as to provide a push notification for the user to put in or take out the appetite control device 1502.

In other embodiments, the output data is associated with appetite information of the user. The appetite information of the user may include such as, user's eating habits, usage of the appetite control device 1502 by the user, mealtimes and snack times for the user and the like.

In some embodiments the system 1500, via the GUI 1510 may learn a user's behavior to identify their appetite information and recommend times for putting in or taking out the appetite control device 1502. The GUI 1502 may also provide the output data in the form of prompts to correct a user behavior. For example, if a user regularly removes the appetite control device 1502 at 8:15 PM for a snack, the system 1500 may provide encouragement and recommend that the user keep the appetite control device 1502 in use to avoid snacking. The system 1500, via the GUI 1510 may also take input from additional devices or user inputs, such as a user weight, a user heart rate, a user number of steps taken, calories taken in, calories burned, or the like. The system 1500 may then determine the output data using the pre-trained ML model. The output is based on the data received from the appetite control device 1502. The system 1500 may use the pre-trained ML model to determine the output. The output may correspond to subsequent action of the user or a food recommendation to the user. The system 1500 may also provide appetite information such as showing any correlation between a loss in weight, lowered resting heart rate, less calories consumed, or the like with increased or proper use of the appetite control device 1502, through the GUI 1510 based on the determined output.

In some embodiments, the GUI 1510 may provide gamification to encourage users to continue use of the appetite control device 1502. For example, the GUI 1510 may track a streak for use of the appetite control device 1502. The GUI 1510 may also encourage the user to ‘beat’ their previous records for use of the appetite control device 1502.

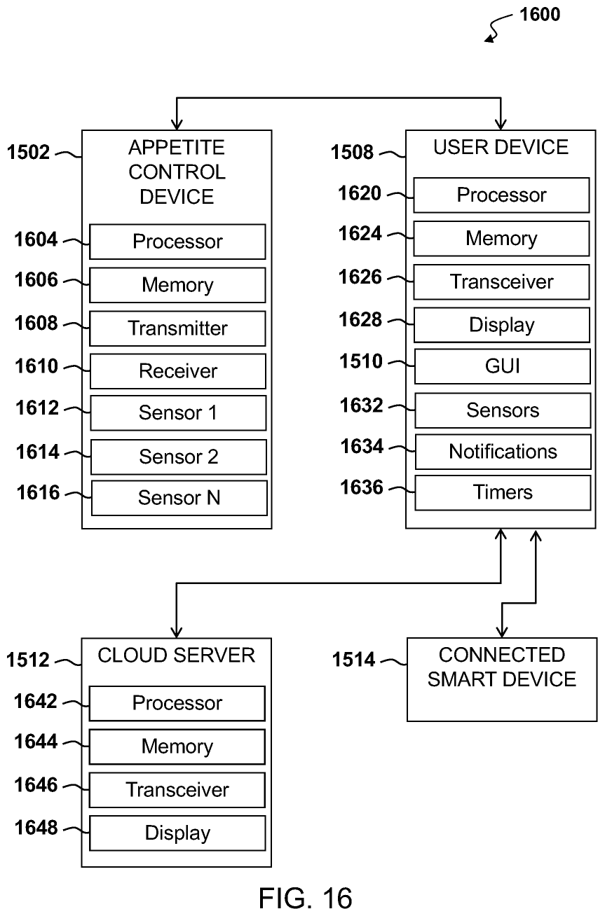

FIG. 16 shows a high-level block diagram of an appetite control device system 1600, in accordance with an example embodiment of the present invention. The appetite control device 1502 may include a processor 1604, memory 1606, a transmitter 1608, a receiver 1610, and one or more sensors 1612, 1614, 1616. In some embodiments, the appetite control device 1502 may include a power source. In some embodiments the appetite control device 1502 may not control any electronics and usage information may be entered by a user into the GUI 1510 and/or receiving from a connected smart device 1514 and/or cloud server 1512. The user device 1508 may be a smartphone, tablet, computer, smartwatch, or the like. The user device 1508 may include a processor 1620, memory 1624, transceiver 1626, display 1628, GUI 1510, one or more sensors 1632, notifications 1634, and timers 1636. The notifications 1634 may be push notifications, text messages, pop-up windows in the GUI 1510, or the like. The timers 1636 may be alarms, countdown timers, usage timers, or the like.

The connected smart device 1514 may be a smartwatch, phone, smart scale, heart rate monitor, or the like. The connected smart device 1514 may provide additional information that may be used with the data from the appetite control device 1502. The cloud server 1512 may include the processor 1642, memory 1644, transceiver 1646, and a display 1648. The cloud server 1512 may store data from the appetite control device 1502. The cloud server 1512 may perform additional calculations, such as training a machine learning model, on the stored data from the appetite control device 1502. For example, the cloud server 1512 may determine habits and recommend notifications 1634 to avoid snacking and encourage use of the appetite control device 1502.

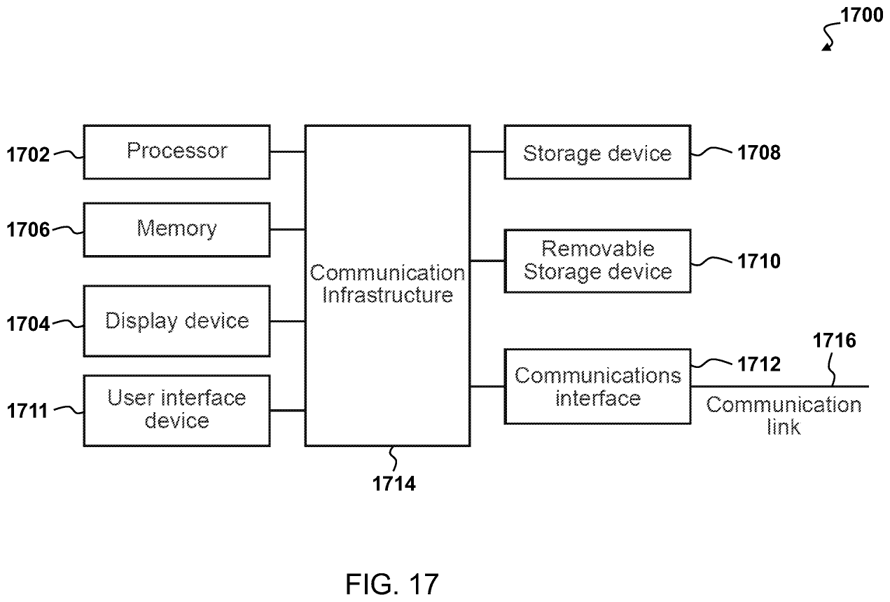

FIG. 17 is a high-level block diagram 1700 showing a computing system comprising a computer system useful for implementing an embodiment of the system and process, disclosed herein. Embodiments of the system may be implemented in different computing environments The computer system includes one or more processors 1702, and can further include an electronic display device 1704 (e.g., for displaying graphics, text, and other data), a main memory 1706 (e.g., random access memory (RAM)), storage device 1708, a removable storage device 1710 (e.g., removable storage drive, a removable memory module, a magnetic tape drive, an optical disk drive, a computer readable medium having stored therein computer software and/or data), user interface device 1711 (e.g., keyboard, touch screen keypad, pointing device), and a communication interface 1712 (e.g., modem, a network interface (such as an Ethernet card), a communications port, or a PCMCIA slot and card). The communication interface 1712 allows software and data to be transferred between the computer system and external devices. The system further includes a communications infrastructure 1714 (e.g., a communications bus, crossover bar, or network) to which the devices/modules are connected as shown. Information transferred via communications infrastructure 1714 may be in the form of signals such as electronic, electromagnetic, optical, or other signals capable of being received by communications infrastructure 1714, via a communication link 1716 that carries signals and may be implemented using wire or cable, fiber optics, a phone line, a cellular/mobile phone link, an radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer implemented process.

Various embodiments have been described with reference to flowchart illustrations and/or block diagrams of methods, apparatus (systems) and computer program products according to embodiments. Each block of such illustrations/diagrams, or combination thereof, can be implemented by computer program instructions The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

In various embodiments, computer programs (i.e., computer control logic) are stored in main memory and/or secondary memory. Computer programs may also be received via a communications interface 1712. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

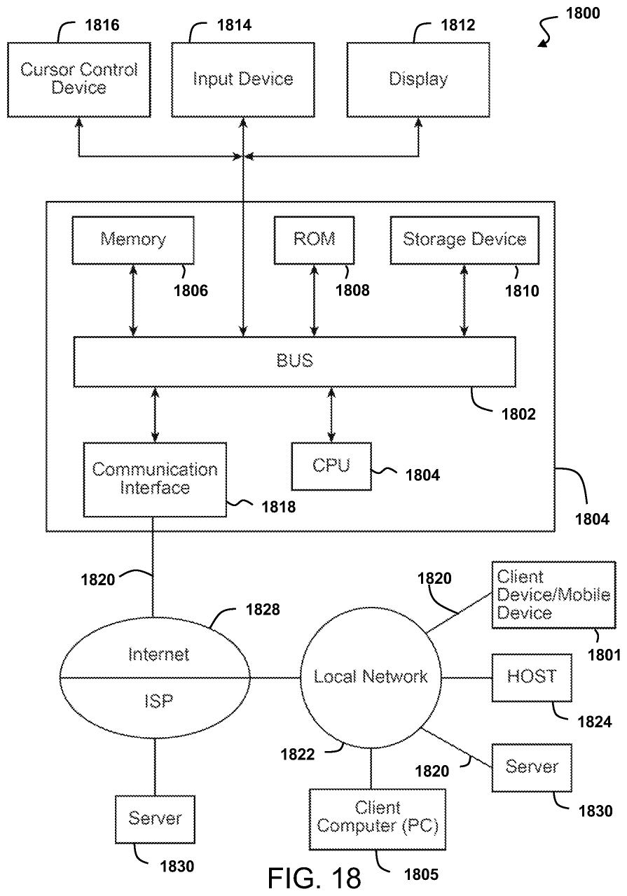

FIG. 18 shows a block diagram of an example system 1800 in which an embodiment may be implemented. The system 1800 includes one or more client devices 1801 such as consumer electronics devices, connected to one or more server computing systems 1830. The server 1830 includes a bus 1802 or other communication mechanism for communicating information, and a processor (CPU) 1804 coupled with the bus 1802 for processing information. The server 1830 also includes a main memory 1806, such as a random access memory (RAM) or other dynamic storage device, coupled to the bus 1802 for storing information and instructions to be executed by the processor 1804. The main memory 1806 also may be used for storing temporary variables or other intermediate information during execution or instructions to be executed by the processor 1804. The server computer system 1830 further includes a read only memory (ROM) 1808 or other static storage device coupled to the bus 1802 for storing static information and instructions for the processor 1804. In some embodiments, a storage device 1810, such as a magnetic disk or optical disk, is provided and coupled to the bus 1802 for storing information and instructions. The bus 1802 may contain, for example, thirty-two address lines for addressing video memory or main memory 1806. The bus 1802 can also include, for example, a 32-bit data bus for transferring data between and among the components, such as the CPU 1804, the main memory 1806, video memory and the storage 1810. Alternatively, multiplex data/address lines may be used instead of separate data and address lines.

The server 1830 may be coupled via the bus 1802 to a display 1812 for displaying information to a computer user. An input device 1814, including alphanumeric and other keys, is coupled to the bus 1802 for communicating information and command selections to the processor 1804. Another type or user input device comprises cursor control 1816, such as a mouse, a trackball, or cursor direction keys for communicating direction information and command selections to the processor 1804 and for controlling cursor movement on the display 1812.

According to one embodiment, the functions are performed by the processor 1804 executing one or more sequences of one or more instructions contained in the main memory 1806. Such instructions may be read into the main memory 1806 from another computer-readable medium, such as the storage device 1810. Execution of the sequences of instructions contained in the main memory 1806 causes the processor 1804 to perform the process steps described herein. One or more processors in a multi-processing arrangement may also be employed to execute the sequences of instructions contained in the main memory 1806. In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor 1804 for execution. Such a medium may take many forms, including but not limited to, nonvolatile media, volatile media, and transmission media. In various embodiments, non-volatile media includes, for example, optical or magnetic disks, such as the storage device 1810. In various embodiments, volatile media includes dynamic memory, such as the main memory 1806. In various embodiments transmission media includes coaxial cables, copper wire and fiber optics, including the wires that comprise the bus 1802. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD ROM, any other optical medium, punch cards, paper tape, any other physical medium with patterns of holes, a RAM, a PROM, an EPROM, a FLASH-EPROM any other memory chip or cartridge, a carrier wave as described hereinafter, or any other medium from which a computer can read.

Various forms of computer readable media may be involved in carrying one or more sequences of one or more instructions to the processor 1804 for execution. For example, the instructions may initially be carried on a magnetic disk of a remote computer. The remote computer can load the instructions into its dynamic memory and send the instructions over a telephone line using a modem. A modem local to the server 1830 can receive the data on the telephone line and use an infrared transmitter to convert the data to an infrared signal. An infrared detector coupled to the bus 1802 can receive the data carried in the infrared signal and place the data on the bus 1802. The bus 1802 carries the data to the main memory 1806, from which the processor 1804 retrieves and executes the instructions. The instructions received from the main memory 1806 may optionally be stored on the storage device 1810 either before or after execution by the processor 1804.

The server 1830 also includes a communication interface 1818 coupled to the bus 1802. The communication interface 1818 provides a two-way data communication coupling to a network link 1820 that is connected to the world wide packet data communication network now commonly referred to as the Internet 1828. The Internet 1828 uses electrical, electromagnetic or optical signals that carry digital data streams. The signals through the various networks and the signals on the network link 1820 and through the communication interface 1818, which carry the digital data to and from the server 1830, are exemplary forms or carrier waves transporting the information.

In another embodiment of the server 1830, interface 1818 is connected to a network 1822 via a communication link 1820. For example, the communication interface 1818 may be an integrated services digital network (ISDN) card or a modem to provide a data communication connection to a corresponding type of telephone line, which can comprise part of the network link 1820. As another example, the communication interface 1818 may be a local area network (LAN) card to provide a data communication connection to a compatible LAN. Wireless links may also be implemented. In any such implementation, the communication interface 1818 sends and receives electrical electromagnetic or optical signals that carry digital data streams representing various types of information.

The network link 1820 typically provides data communication through one or more networks to other data devices. For example, the network link 1820 may provide a connection through the local network 1822 to a host computer 1824 or to data equipment operated by an Internet Service Provider (ISP). The ISP in turn provides data communication services through the Internet 1828. The local network 1822 and the Internet 1828 both use electrical, electromagnetic or optical signals that carry digital data streams. The signals through the various networks and the signals on the network link 1820 and through the communication interface 1818, which carry the digital data to and from the server 1830, are exemplary forms or carrier waves transporting the information.

The server 1830 can send/receive messages and data, including e-mail program code, through the network, the network link 1820 and the communication interface 1818. Further, the communication interface 1818 can comprise a USB/Tuner and the network link 1820 may be an antenna or cable for connecting the server 1830 to a cable provider, satellite provider or other terrestrial transmission system for receiving messages, data and program code from another source.

The example versions of the embodiments described herein may be implemented as logical operations in a distributed processing system such as the system 1800 including the servers 1830. The logical operations of the embodiments may be implemented as a sequence of steps executing in the server 1830, and as interconnected machine modules within the system 1800. The implementation is a matter of choice and can depend on performance of the system 1800 implementing the embodiments. As such, the logical operations constituting said example versions of the embodiments are referred to for e.g., as operations, steps or modules.

Similar to a server 1830 described above, a client device 1801 can include a processor, memory, storage device, display, input device and communication interface (e.g., e-mail interface) for connecting the client device to the Internet 1828 the ISP, or LAN 1822, for communication with the servers 1830.

The system 1800 can further include computers (e.g., personal computers, computing nodes) 1805 operating in the same manner as client devices 1801, wherein a user can utilize one or more computers 1805 to manage data in the server 1830.

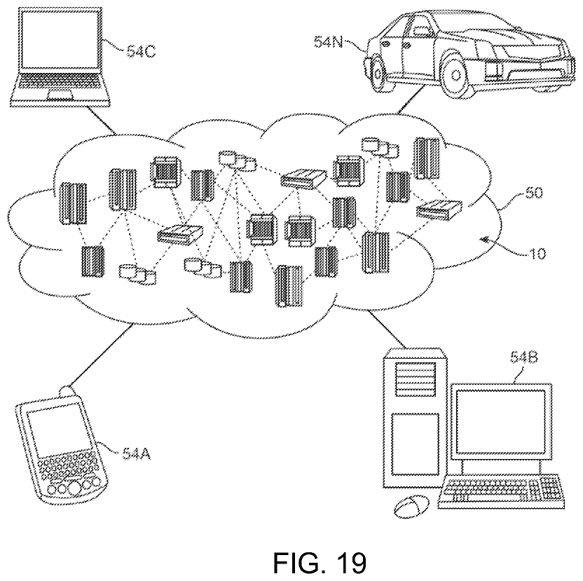

Referring to FIG. 19, illustrative cloud computing environment 50 for the system 1500 is depicted. As shown, cloud computing environment 50 comprises one or more cloud computing nodes 10 with which local computing devices used by cloud consumers, such as, for example, personal digital assistant (PDA), smartphone, smart watch, set-top box, video game system, tablet, mobile computing device, or cellular telephone 54A, desktop computer 548, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 19 are intended to be illustrative only and that computing nodes 10 and cloud computing environment 50 can communicate with any type of computerized device over any type of network and/or network addressable connection (e.g., using a web browser).

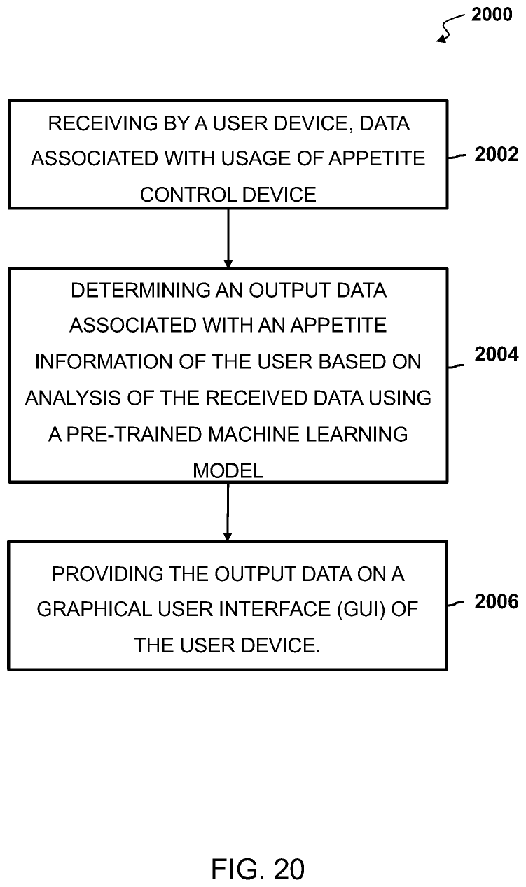

FIG. 20 shows a computer implemented method 2000 for providing appetite information of user, in accordance with an example embodiment of the present invention. The method 2000 may be executed by the system 1500 to provide an output related to appetite information of the user.

At step 2002, data associated with usage of appetite control device 1502 is received by a user device 1508. The data may be gathered by a plurality of sensors (1504, 1506) connected with the appetite control device 1502. The data may comprise information regarding time worn, time in movement, amount of movement, any ‘chewing’ or ‘grinding’ movements, number of uses, liquid detection, food detection, or the like.

At step 2004, an output data associated with appetite information of the user is determined. The output is determined based on analysis of the received data at step 2002 using a pre-trained machine learning (ML) model. The ML model may be used for analyzing eating patterns of the user and finding correlation between the eating pattern and diseases that are prone to the user. In an embodiment, the method 2004 may anonymize user data and transmit the anonymized user data to the cloud server 1512. The method 2004 may also obtain the anonymized user data from multiple users and perform analysis using the pre-trained ML model in the cloud server 1512. For example, the pre-trained ML model performs the analysis using user's historical appetite information using deterministic algorithm, stochastic algorithm or combination thereof, such as, Bayesian network, neural network, fuzzy logic, and the like.

At step 2006, the output data is provided on a Graphical User Interface (GUI) of the user device 1508. The method 2006 may provide the output data as a recommendation to the user, based on the analysis using the ML model. The ML model may use the analyzed historical appetite information to further learn pattern. The pattern may be learnt based on one or more ML models that may include, but not limited to, a Convolutional Neural Network (CNN) model, a Hidden Marrkov Model (HMM), or a Recurrent Neural Network (RNN) model. The pre-trained ML model may also use pattern recognition algorithms. The pattern recognition algorithms may include classification methods, clustering methods, ensemble learning methods, multi-linear space learning methods, regression methods, labeling methods or the like. The pattern recognition algorithms may help in identifying behavior and trends of user's appetite information. The output provided as a recommendation to the user may be one or more of alerts or prompts or timing information that ensures the user wears the appetite control device 1502. In some embodiments, output may be a recommendation of specialized food to a user. The specialized food may help the user to maintain stable metabolism and reduce weight.

Those skilled in the art will appreciate that various adaptations and modifications of the described preferred embodiments can be configured without departing from the scope and spirit of the improved pressure switch system described herein. Therefore, it is to be understood that, within the scope of the embodiments, the switch system may be practiced other than as specifically described herein.

What is claimed is:

1. A system comprising:
an appetite control device for covering a plurality of teeth, wherein the appetite control device comprises an arcuate shape having an outer sidewall, an inner sidewall and a top wall connecting the inner sidewall and the outer sidewall to form a channel, wherein the channel is configured for receiving a plurality of teeth, wherein the appetite control device comprises a larger width at ends and a narrower width at a front of the appetite control device; and
a user device comprising:
a memory configured to store executable instructions; and
a processor configured to execute the executable instructions stored in the memory, the processor configured to:
receive, data associated with usage of the appetite control device;
determine, an output data associated with appetite information of the user based on analysis of the received data using a pre-trained machine learning model; and
provide, the output data on a Graphical User Interface (GUI) of the user device.

2. The system of claim 1, wherein the appetite control device comprises one or more apertures on at least one of the outer side wall, the inner side wall, and the top wall.

3. The system of claim 1, wherein the appetite control device comprises a flavour pouch comprising one or more flavour tabs.

4. The system of claim 3, wherein the flavour pouch comprises a top surface and a bottom surface to hold the one or more flavour tabs.

5. The system of claim 3, wherein the flavour pouch comprises a plurality of apertures with varying sizes.

6. The system as claimed in claim 3, wherein the flavour pouch is made from a mesh to hold the one or more flavour tabs.

7. The system as claimed in claim 3, wherein the flavour pouch is attached to the appetite control device by one or more attachment fasteners.

8. The system as claimed in claim 3, wherein the flavour pouch comprises surface indicia to provide feedback to the user.

9. The system of claim 1, wherein the appetite control device comprises a front portion of the outer side wall to cover a part of front portion of the plurality of teeth of the user.

10. The system of claim 1, wherein the appetite control device comprises a portion of the top wall to cover a part of top portion of the plurality of teeth of the user.

11. The system of claim 10, wherein the portion comprises one or more of a hook or a J shaped portion to secure against the plurality of teeth of the user.

12. The system of claim 1, wherein the appetite control device comprises an upper appetite control device to cover upper teeth of the user and a lower appetite control device to cover lower teeth of the user, wherein the lower appetite control device is identical to the upper appetite control device.

13. The system of claim 1, wherein the appetite control device is made of mouldable material to ergonomically align with the plurality of teeth of the user.

14. The system of claim 1, wherein the appetite control device comprises a plurality of sensors to provide the data associated with usage of the appetite control device to the user device.

15. The system of claim 1, wherein the processor is further configured to:
provide gamification elements, via the GUI, to the user based on the received data.

16. The system of claim 1, wherein the system further comprises a server, wherein the server is one or more of a physical server or a cloud server.

17. The system of claim 16, wherein the machine learning model is processed in one or more of:
the user device the physical server, or the cloud server.

18. A computer-implemented method comprises:
receiving, by a user device, data associated with usage of an appetite control device;
analyzing, by a pre-trained machine learning model, the received data associated with usage of the appetite control device;
determining, an output data associated with appetite information of the user based on analysis of the received data; and
providing, the output data on a Graphical User Interface (GUI) of the user device.

19. The method of claim 18, wherein receiving data associated with usage of the appetite control device further comprises receiving the data from a plurality of sensors associated with the appetite control device to provide the data associated with usage of the appetite control device to the user device.

20. The method of claim 18, wherein the method further comprises providing gamification elements, via the GUI, to the user based on the received data.
